(19) United States
(12) Patent Application Publication
(10) Pub. No.: US 2004/0254238 A1
(43) Pub. Date: Dec. 16, 2004

(54) Bone growth stimulation with NO/statin and other NO modulating combinations

(75) Inventors: I. Ross Garrett, San Antonio, TX (US); Gregory R. Mundy, San Antonio, TX (US); Gloria Gutierrez, San Antonio, TX (US)

Correspondence Address: MORRISON & FOERSTER LLP, 3811 VALLEY CENTRE DRIVE, SUITE 500, SAN DIEGO, CA, 92130-2332, US

(73) Assignee: OSTEOSCREEN, San Antonio, TX (US)

(21) Appl. No.: 10/820,565

(22) Filed: Apr. 7, 2004

Related U.S. Application Data
(60) Provisional application No. 60/461,317, filed on Apr. 7, 2003.
(60) Provisional application No. 60/504,095, filed on Sep. 19, 2003.
(60) Provisional application No. 60/513,771, filed on Oct. 22, 2003.

(51) Int. Cl. A61K 31/366; A61K 31/225

(52) U.S. Cl. 514/460; 514/548

(57) ABSTRACT

Methods and compositions provided herein relate to the promotion of bone formation and are thus useful in treating osteoporosis, bone fracture or deficiency, primary or secondary hyperparathyroidism, periodontal disease or defect, metastatic bone disease, osteolytic bone disease, post-plastic surgery, post-prosthetic joint surgery, and post-dental implantation. Disclosed is a method of enhancing bone formation by administering at least two components selected from at least one statin-like compound, at least one nitric oxide generating system, and at least one phosphodiesterase inhibitor. Also disclosed is a pharmaceutical composition comprising said at least two components.

### CROSS-REFERENCE TO RELATED APPLICATIONS

This application claims the benefit of U.S. Provisional Application Ser. No. 60/461,317 filed Apr. 7, 2003, U.S. Provisional Application Ser. No. 60/504,095 filed Sep. 19, 2003, and U.S. Provisional Application Ser. No. 60/513,771 filed Oct. 22, 2003 under 35 U.S.C. § 119 (e). The entire contents of all of these applications are fully incorporated herein by reference.

### TECHNICAL FIELD

Methods and compositions to stimulate bone growth and useful in treating bone disorders in vertebrates, including fractures and cartilage disorders are described. More specifically, the methods and compositions relate to the administration of at least two of 1) a statin-like compound, 2) an NO generating system and 3) a phosphodiesterase (PDE) inhibitor to promote bone growth.

### BACKGROUND

Bone is a dynamic structure characterized by continuous remodeling throughout life. Bone remodeling consists of the coupled processes of bone resorption and bone formation. During the resorptive process, osteoclasts degrade and remove bone. Osteoclasts derive from the monocyte-macrophage lineage and fuse to form mature multi-nucleated cells upon activation by RANK-ligand. Osteoblasts, on the other hand, produce and secrete an extracellular matrix, osteoid, that calcifies to form new bone during the bone formation process as well as a number of enzymes and structural proteins of the bone matrix, including Type-1 collagen, osteocalcin, osteopontin and alkaline phosphatase. Osteoblasts also synthesize a number of growth regulatory peptides, including bone morphogenic proteins (BMPs). Numerous other cytokines, growth factors, and hormones influence the activities of osteoclasts and osteoblasts during remodeling, including IL-1, TNF, TGF-β, and estrogen.

Bone remodeling facilitates the maintenance of healthy bone as well as the repair of any deficits in the bone. The need to enhance bone formation characterizes a variety of conditions associated with bone defects or deficits. For example, the stimulation of bone growth after a bone fracture would hasten and complete the bone repair. Similarly, agents enhancing bone formation are useful in facial reconstruction procedures. Other bone deficit conditions benefiting from increased bone formation include bone segmental defects, periodontal disease, metastatic bone disease, and osteolytic bone disease. Conditions needing connective tissue repair (e.g., healing or regeneration of cartilage defects or injury) would also benefit from enhanced bone formation. As another example, the high fracture risk characterizing osteoporosis associated with age and post-menopausal hormone status can potentially be lessened or ameliorated with bone formation inducing agents. Likewise, primary and secondary hyperparathyroidism, disuse osteoporosis, diabetes-related osteoporosis, and glucocorticoid-related osteoporosis are characterized by the need for bone growth.

To date, therapies and compounds attempting to enhance bone growth target the resorptive process mediated by osteoclasts. For example, Labroo, et al., disclose compounds described as useful in the treatment of osteoporosis in U.S. Pat. No. 5,280,040. These compounds putatively reduce bone loss associated with osteoporosis by preventing bone resorption. Similarly, bisphosphates inhibit the resorption of bone. Bisphosphonates or the methylene bisphosphonic acids are comprised of two phosphonic acid residues coupled through a methylene linkage. Typical representatives include clodronate, ibandronate, risedronate, alendronate and pamidronate. These compounds appear to mediate the inhibition of bone resorption by affecting the apoptosis of osteoclasts. Luckman, S. P., et al., J Bone Min Res (1998) 13:581-589.

Lipid clearing agents represent another class of compounds identified as inhibitors of bone resorption. Certain lipid clearing agents, exemplified by lovastatin and bezafibrate, inhibit bone resorption induced by steroid administration. Wang, G.-J., et al., J Formos Med Assoc. (1995) 94:589-592. Steroid-induced bone loss is associated with a decrease in bone formation attributed to an inhibitory effect of corticosteroid on osteoblast activity coupled with an increase in bone absorption due to direct osteoclast stimulation and an indirect inhibition of intestinal calcium absorption with a secondary increase in parathyroid hormone production. Other mechanisms implicated in steroid-mediated bone loss include those attributable to lipid abnormalities and hyperlipidemia leading to circulatory impairment, obstruction of subchondral vessels, osteocyte necrosis and osteoporosis. In this particular study, the authors attribute the effect on bone loss to their ability to lower lipid levels and overcome the impairment to circulation within the femoral head, given the known activities of lovastatin and bezafibrate. The direct enhancement of bone formation by lipid clearing agents is not addressed.

Lipid clearing agents alone do not normally stimulate bone formation. Wang (1995) supra. Steroids induce triglyceride vesicles in osteoprogenitor cells, reducing the maintenance of theosteogenicphenotype. Cui, Q., et al., J. Bone Mineral Res. (1996) 11(S1):S510. In this report, the lipid clearing agent, lovastatin diminished triglyceride vesicles that accumulated in osteoprogenitor cells treated with dexamethasone in vitro, thereby allowing the cells to maintain the osteogenic phenotype after dexamethasone treatment. However, the direct enhancement of bone formation in the absence of steroid treatment was not addressed.

The inhibition of bone resorption alone slows the rate of bone loss, but does not increase the rate of bone formation. In point of fact, anti-resorptive agents ultimately decrease the rate of bone formation. Marcus, R., Agents Affecting Calcification and Bone Turnover, in THE PHARMACOLOGICAL BASIS OF THERAPEUTICS 1519 (Hardman, et al. eds., 1996). Simply stated, the resorptive and formation processes, though coupled, are distinct and non-overlapping. Therefore, an agent may affect one process and have no effect on the other. The independence of these processes has been confirmed in in vivo models of bone formation. For example, Ducy, P., et al., Nature (1996) 382:448-452 reported that osteocalcin deficient mice exhibit a phenotype marked by increased bone formation and bones of improved functional quality, without impairment of bone resorption. Because inhibitors of bone resorption ultimately result in bone loss, methods for stimulating bone formation must employ agents that enhance or stimulate the bone formation process itself.

One group of compounds suggested for enhancing bone formation are bone morphogenic proteins (BMPs). The BMPs are novel factors in the extended transforming growth factor 13 superfamily. Recombinant BMP-2 and BMP-4 can induce new bone formation when they are injected locally into the subcutaneous tissues of rats. Wozney, J., Molec Reprod Dev (1992) 32:160-167. Normal osteoblasts express these factors as they differentiate. Furthermore, BMPs stimulate osteoblast differentiation and bone nodule formation in vitro as well as bone formation in vivo. Harris, S., et al., J. Bone Miner Res (1994) 9:855-863. This latter property suggests potential usefulness as therapeutic agents in diseases that cause or result in bone loss. While BMPs act as potent stimulators of bone formation in vitro and in vivo, BMP receptor expression is not restricted to the bone, indicating potential side effects in other tissues with exogenous systemic BMP administration. This lack of specificity may impose limitations to the development of BMPs as therapeutic agents.

However, the recognition of BMPs as inducers of bone formation provides an avenue for the identification of other agents useful in stimulating bone growth. Using a BMP-reporter assay, small molecules have been identified as potentially useful in treating bone disorders in vertebrates. For example, compounds with the general formula Ar¹—L—Ar² wherein Ar¹ and Ar² are aromatic moieties and L is a linker that separates them be a specified distance enhanced the expression of the BMP-reporter construct. See PCT Application No. WO 98/17267 (published 30 Apr. 1998). Similar compounds are disclosed for this purpose in earlier filed PCT Application Nos. WO 97/15308 (published 1 May 1997) and WO 97/48694 (published 24 Dec. 1997).

Statins per se are generally understood to be HMG-CoA reductase inhibitors identified as anabolic for bone. See Mundy, et al., U.S. Pat. Nos. 6,022,887, 6,080,779 and 6,376,476. HMG-CoA reductase is the principal rate limiting enzyme involved in cellular cholesterol biosynthesis. The pathway is also responsible for the production of dolichol, ubiquinones, isopentenyl adenine and farnesol. HMG-CoA reductase converts 3-hydroxy-3-methyl-glutaryl CoA (HMG-CoA) to mevalonate. Statins or HMG-CoA reductase inhibitors have pleiotropic effects that include lowering serum cholesterol, modifying endothelial cell functions, inflammatory responses, smooth muscle activation, atherosclerotic plaque stability, and thrombus formation.

NO is another compound with a wide spectrum of physiologic functions. NO inhibits platelet aggregation and adhesion, mediates glutamate neurotoxicity, relaxes smooth muscles, and is cytotoxic and cytostatic for microorganisms. Fukuto, J. M., et al., Ann. Rev. Pharmacol. (1995) 35:165-194. On the other hand, increased production of NO is also associated with pathophysiology in almost every organ system, either alone or in the presence of other free radicals. Vallance, P., et al., Nature Rev. Drug Dis. (2002 1:839-850). Thus, depending on the physiological circumstances, either an increase or a decrease in NO production may be desirable. For example, NO can promote or inhibit apoptosis, eliminate tumors or augment their metastatic or vascularization potential, as well as increase and protect against damage after stroke. Vallance (2002), supra.

Numerous compounds have been identified that modulate NO production. For example, L-arginine as the substrate of NO synthase (NOS) enhances the production of NO. NOS exists as one of three isoforms that catalyze the conversion of L-arginine to nitric oxide (NO) and citrulline. Hobbs, A. J., et al., Ann. Rev. Pharmacol. (1999) 39:191-220. Another source of NO is organic nitrates. In particular, glyceryl trinitrate (commonly known as nitroglycerin) acts as a substrate for various enzymes including mitochondrial aldehyde dehydrogenase (mtALDH). Chen, Z., et al., Proc. Nat'l. Acad. Sci. U.S.A. (2002) 99:8306-8311. The mtALDH-mediated conversion of glycerol trinitrate yields 1,2-glycerol dinitrate and NO.

Cyclic AMP (cAMP) and cyclic GMP (cGMP) are important as second messengers in various signaling pathways. Phosphodiesterases (PDE) catalyze the conversion of cAMP or cGMP to 5′-AMP or 5′GMP and thus control the duration and amplitude of the cAMP or cGMP signal. There are eleven families of phosphodiesterases that have been characterized including calcium/calmodulin dependent, cGMP-stimulated, cGMP-inhibited cAMP-specific and cGMP-specific.

A recent article by Essayan, D. M., J. Allergy Clin. Immunol. (2001) 108:671-680, incorporated herein by reference, is a helpful summary of the subtypes reflecting the current state of knowledge concerning the various PDE's. Table 1 in that article lists the eleven known families; it is indicated that additional families are postulated but that insufficient data for these additional families is available to include them. Each family contains a number of members, and the number of specific human PDE's is estimated to be >50. The article further confirms that nonselective inhibitors of phosphodiesterases include caffeine, theophylline, pentoxifylline and 3-isobutyl-1-methylxanthine.

Horiuchi, H., et al., Bone (2001) 28:290-294 and Kinoshita, T., et al., Bone (2000) 27:811-817 have shown that pentoxifylline, which is a nonspecific phosphodiesterase inhibitor and rolipram, which is a PDE-4 inhibitor, increase bone mass by promoting bone formation. An additional article reports on a methylxanthine derivative's effect on osteoporosis. Robin, J. C., et al., J. Med. (1983) 14:137-145. Also reporting an effect of a nonspecific phosphodiesterase inhibitor on bone formation is an article by Rawadi, G., et al., Endocrinology (2001) 142:4673-4682. The effectiveness of an additional inhibitor of PDE-4, XT-44 has also been disclosed by Waki, Y, et al., Jpn J. Pharmacol. (1999) 79:477-483 and the effect of cAMP accumulation in osteoblast cells has been described by Ahlstrom, M., et al., Biochem. Pharmacol. (1999) 58:1335-1340. cAMP is a substrate for PDE-4, PDE-7, and PDE-8.

Finally, Wakabayashi, S., describes the effect of several selective inhibitors on osteoblast cell differentiation in an article in J Bone Min. Res. (2000) 15:Suppl. 1, Abstract M198. In addition, U.S. Pat. No. 6,010,711 describes phosphodiesterase inhibitors which also inhibit interleukin-1, interleukin-6, and tumor necrosis factor α. This class of compounds includes nonspecific phosphodiesterase inhibitors as well as rolipram, a selective PDE-4 inhibitor.

PDE-5 is a cGMP specific phosphodiesterase which is found primarily in lung, platelets, and smooth muscle. Inhibitors of PDE-5 include dipyridamole, MY-5445, sildenafil (Viagra®) and Zaprinast™. Dipyridamole and Zaprinast also inhibit PDE-6.

The present application discloses methods, and compositions, for stimulating bone formation that employ combinations of at least two types of agents that modulate NO-involved systems. These agents are: first, statin-like compounds, including statins per se, second, systems involved in the generation of nitric oxide (NO), and third, phosphodiesterase inhibitors.

### DISCLOSURE OF THE INVENTION

The present invention employs combinations of agents that are relevant to the physiological functions of nitric oxide (NO). It is understood that one category of such agents includes statin-like compounds, including the statins per se. Another set of such agents includes those that result in the generation of nitric oxide; a third set of agents is the phosphodiesterase inhibitors, as these compounds interfere with the ability of the phosphodiesterases to control the signals mediated by cyclic AMP and cyclic GMP, cyclized nucleotide monophosphates that are generated in response to NO signaling. Of course, all three of these types of NO pathway-related agents may be used.

Thus, in one aspect, the invention is directed to methods and compositions that stimulate the growth or repair of skeletal tissue which employ a combination of at least two of

(a) at least one nitric oxide (NO) generating system;

(b) at least one statin-like compound; and

(c) at least one phosphodiesterase (PDE) inhibitor.

The nitric oxide generating system is illustrated by, but is not limited to, substrates such as organic nitrates and L-arginine, and enzymes that convert substrates to NO. The nature of NO-generating systems is further described hereinbelow. These systems may also include stimulators of NO synthase activity and production.

Statin-like compounds are characterized by virtue of their ability to inhibit steps in the isoprenoid/steroid synthesis pathway. Some statin-like compounds include statins per se, i.e., those of the formulas:

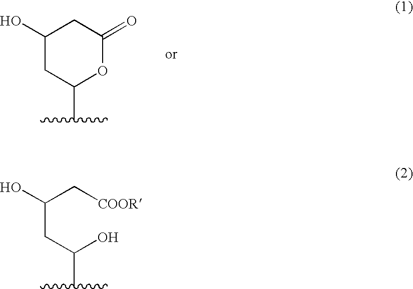

wherein R′ represents a cation, H or a substituted or unsubstituted alkyl group of 1-6C;

wherein the lactone or open lactone is coupled to an organic moiety of up to 40C.

In many statins, the indicated bond in formulas (1) and (2) is coupled to X-Y

wherein X represents substituted or unsubstituted alkylene (1-6C), alkenylene (2-6C), or alkynylene (2-6C); and

Y represents one or more carbocyclic and/or heterocyclic rings; when two or more rings are present in Y, they may optionally be fused.

Statin-like compounds also include those which are effective at points in the isoprenoid/steroid synthesis pathway not necessarily involving the targets of the statins per se (i.e., HMG-CoA reductase) and may not share the structural features of formulas (1) and (2) shown above. Such agents include, for example, apamine and zaragozic acid.

The third type of agent relevant to NO mediated pathways is a phosphodiesterase (PDE) inhibitor. A number of such inhibitors are known in the art, including non-specific inhibitors such as theophylline and caffeine as well as inhibitors that are specific for one or more of the PDE's, such as sildenafil.

The statin-like compound is administered in combination with the nitric oxide generating system or in combination with the PDE inhibitor, or the nitric oxide-generating system is administered in combination with the PDE inhibitor or all three types of agents are administered, either simultaneously (e.g., in a single composition) or separately to stimulate bone growth.

In another aspect, a pharmaceutical composition is provided in unit dosage form to enhance bone formation in a vertebrate animal, which composition comprises a pharmaceutically acceptable excipient and an amount, effective to promote bone formation, of at least two of a) a nitric oxide generating system, b) a statin-like compound, and c) a PDE inhibitor.

The methods and compositions of the invention may involve all three of a statin-like compound, a nitric oxide-generating system and a PDE inhibitor. In addition, the methods and compositions may include one or more additional agents that stimulate bone formation and/or inhibit bone resorption.

Exemplary of such agents that can be used in the combination include bone morphogenetic factors, anti-resorptive agents, osteogenic factors, cartilage-derived morphogenetic proteins, growth hormones, estrogens, bisphosphonates, differentiating factors, compounds that inhibit activity of proteasomal activity, e.g., antibodies that specifically bind to proteasomal proteins, and compounds that inhibit production of a proteasomal protein, e.g., antisense oligos that are complementary to genes or RNA's that encode proteasomal proteins or inhibitory RNA (iRNA) constructs. For clinical uses, the antibodies are preferably monoclonal or humanized antibodies.

### BRIEF DESCRIPTION OF THE DRAWINGS

FIGS. 1A and 1B show the structures and activity of several compounds of the disclosed methods and compositions.

FIG. 2 shows an outline of the synthesis pathway for isoprenoids and the pathways of their subsequent conversion to squalene and steroids and in prenylating target proteins.

FIG. 3 shows the effect of L-arginine on statin-stimulated bone formation in the neonatal murine calvarial assay. The left panel depicts bone formation observed after treatment with simvastatin at the doses indicated. The right panel depicts bone formation observed after treatment with 20 μM L-arginine with simvastatin at the doses indicated.

FIG. 4 shows the in vivo effects of L-arginine administered with OsteoPure™ by oral gavage. The upper panel shows the bone mineral densities (BMDs) expressed as gm/cm². The lower panel shows the % change in each group from the beginning of the experiment up to 16 weeks of treatment. Statin equivalence: 100 mg/kg/day=0.72 mg/day; 400 mg/kg/day=2.9 mg/day; and 800 mg/kg/day=5.8 mg/day.

### MODES OF CARRYING OUT THE INVENTION

The ultimate goal of the methods and compositions of the invention is to treat or ameliorate bone disorders in vertebrate subjects, particularly mammals, and more particularly humans.

Stimulation of Bone Growth

Generally, the methods and compositions provided herein may be used to stimulate growth of bone-forming cells or their precursors, or to induce differentiation of bone-forming cell precursors in vitro, ex vivo or in vivo. The compounds described herein may also modify a target tissue or organ environment, so as to attract bone-forming cells to an environment in need of such cells, thereby stimulating bone growth.

As used herein, the term “precursor cell” or “cell precursor” refers to a cell that is committed to a differentiation pathway, but that generally does not express markers or function as a mature, fully differentiated cell. The term “mesenchymal cells” or “mesenchymal stem cells” refers to pluripotent progenitor cells that are capable of dividing many times, and whose progeny will give rise to skeletal tissues, including cartilage, bone, tendon, ligament, marrow stroma and connective tissue. See Caplan, A., J. Orthop. Res. (1991) 9:641-650. “Osteogenic cells” include mature osteoblasts, differentiating osteoblasts, and osteoblast precursor cells.

In one embodiment, the methods and compositions provided can be used for stimulating a cell population containing marrow mesenchymal cells, thereby increasing the number of osteogenic cells within the population. Stimulation by the methods of the invention may be conducted ex vivo. In one embodiment, hematopoietic cells are removed from the cell population, either before or after stimulation by the disclosed methods and expanded. The expanded osteogenic cells can then be infused (or reinfused) into a vertebrate subject in need thereof. For instance, a subject's own mesenchymal stem cells can be treated using the disclosed methods ex vivo, and the resultant osteogenic cells could be infused or directed to a desired site within the subject, where further proliferation and/or differentiation of the osteogenic cells can occur without immunorejection. Alternatively, the cell population treated by the disclosed methods may be immortalized human fetal osteoblastic or osteogenic cells. If such cells are infused or implanted in a vertebrate subject, it may be advantageous to “immunoprotect” these non-self cells, or to immunosuppress (preferably locally) the recipient to enhance transplantation and bone or cartilage repair.

In another embodiment, the methods and compositions thereof, stimulate bone formation in vivo in a subject in need thereof. The compositions are administered systemically or locally, simultaneously or separately, over any necessary period of time. The anabolic increase in bone formation can result from increasing the absolute number, differentiation state, activation state, metabolic activity, or lifespan of the osteoblast directly or indirectly, or any combination thereof.

In the methods provided herein, an “effective amount” of the compositions is that amount which produces a statistically significant anabolic effect on the bone. For example, an “effective amount” for therapeutic uses is the amount of the compositions comprising the active compounds required to provide a clinically significant increase in healing rates in fracture repair; reversal of bone loss in osteoporosis; reversal of cartilage defects or disorders; prevention or delay of onset of osteoporosis; stimulation and/or augmentation of bone formation in fracture non-unions and distraction osteogenesis; increase and/or acceleration of bone growth into prosthetic devices; and repair of dental defects. Such effective amounts will be determined using routine optimization techniques and are dependent on the particular condition to be treated, the condition of the patient, the route of administration, the formulation, and the judgment of the practitioner and other factors evident to those skilled in the art. The dosage useful in the invention (e.g., in osteoporosis) is manifested as a statistically significant difference in bone mass between treatment and control groups (i.e., groups not received the composition(s) containing the active ingredients). This difference in bone mass may be seen, for example, as a 5-20% or more increase in bone mass in the treatment group relative to the control group. Other measurements of clinically significant increases in healing may include, for example, tests for breaking strength and tension, breaking strength and torsion, 4-point bending, increased connectivity in bone biopsies and other biomechanical tests well known to those skilled in the art. General guidance for treatment regimens is obtained from experiments carried out in animal models of the disease of interest.

Bone Deficits and Disorders

Any bone deficit or disorder improved or remedied by increased bone formation can be treated using the methods and compositions of the invention. Exemplary conditions appropriate for treatment with the disclosed methods and compositions herein include: repair of bone defects and deficiencies, such as those occurring in closed, open and non-union fractures; prophylactic use in closed and open fracture reduction; promotion of bone healing in plastic surgery; stimulation of bone in growth into non-cemented prosthetic joints and dental implants; elevation of peak bone mass in pre-menopausal women; treatment of growth deficiencies; treatment of periodontal disease and defects, and other tooth repair processes; increase in bone formation during distraction osteogenesis; and treatment of other skeletal disorders, such as age-related osteoporosis, post-menopausal osteoporosis, glucocorticoid-induced osteoporosis or disuse osteoporosis and arthritis, or any condition that benefits from stimulation of bone formation. The methods and compositions provided herein can also be useful in repair of congenital, trauma-induced or surgical resection of bone (i.e., for cancer treatment), and in cosmetic surgery. Further, the present methods and compositions can be used for limiting or treating cartilage defects or disorders, as well as in wound healing or tissue repair.

Any subject can be treated with the invention methods and compositions. Such a subject is a mammal, preferably a human, with a bone disorder or disease or a condition that would benefit from the stimulation of bone growth. Veterinary uses of the disclosed methods and compositions are also contemplated. Such uses would include treatment of bone or cartilage deficits or defects, i.e., bone disorders, in domestic animals, livestock and thoroughbred horses.

As used herein, “treat” or “treatment” includes a postponement of development of bone deficit symptoms and/or a reduction in the severity of such symptoms that will or are expected to develop. The terms further include ameliorating existing bone or cartilage deficit symptoms, preventing additional symptoms, ameliorating or preventing the underlying metabolic causes of symptoms, preventing or reversing bone resorption and/or encouraging bone growth. Thus, the terms denote that a beneficial result has been conferred on a vertebrate subject with a cartilage, bone or skeletal deficit, or with the potential to develop such deficit.

As used herein, the term “bone deficit” refers to an imbalance in the ratio of bone formation to bone resorption, such that, if unmodified, the subject will exhibit less bone than desirable, or the subject's bones will be less intact and coherent than desired. Bone deficit may also result from trauma (e.g., fracture), surgical intervention, dental or periodontal disease, hormonal imbalance, bone disease, non-bone disease states (e.g., cancer), congenital or genetic deficiencies, or drug therapy (e.g., chemotherapy).

As used herein, the term “cartilage defect” refers to damaged cartilage, less cartilage than desired, or cartilage that is less intact and coherent than desired. Cartilage deficit may result from trauma (e.g., fracture), surgical intervention, dental or periodontal disease, hormonal imbalance, bone disease, non-bone disease states (e.g., cancer), congenital or genetic deficiencies, or drug therapy (e.g., chemotherapy).

As used herein, the term “bone disorders” includes both bone deficits and cartilage defects.

Nitric Oxide Generating Systems

The nitric oxide generating system encompasses any compound, enzyme or substrate that stimulates, enhances, or participates in the formation of nitric oxide.

Nitric oxide production from arginine is mediated by several isoforms of nitric oxide synthase, including isoform I (nNOS), isoform II (iNOS), and isoform III (eNOS). Isoforms I and III are Ca⁺²/calmodulin-requiring, constitutive enzymes present in neural tissue and vascular endothelial cells respectively; isoform II is calcium independent and is inducible by mediators of inflammation. Nitric oxide formation from inorganic nitrates such as nitroglycerin is mediated by mitochondrial aldehyde dehydrogenase (mtALDH) or can occur spontaneously without enzyme catalysis. Systems from which NO can be generated useful in the invention methods may include these enzymes or means to modulate their activity or production, and/or may include L-arginine and other nitric oxide donors such as nitroprusside, nitrosothiol, and organic nitrates. Formation of nitric oxide can sometimes require the presence of additional compounds such as cysteine and glutathione, and these may be included in the system as well.

Thus, as defined, a “nitric oxide generating system” is any compound or combination of compounds, including enzymes or nucleic acids encoding enzymes, that results in production of or enhancement of production of NO. At the simplest level, these systems provide compounds that are themselves the origin of NO (i.e., “NO donors”) such as arginine and organic nitrates. NO generating systems may also be cells modified to produce enzymes that catalyze these conversions such as the various isoforms of NOS especially eNOS or mtALDH. The cells may be modified with recombinant production systems for these enzymes. The enzymes themselves may be administered as well as reaction mixtures comprising the enzymes and their substrates. In addition, these systems can comprise compounds that are associated with NO production such as thiol pools which contain cysteine or glutathione, compounds that stimulate the activity of the relevant enzymes or compounds that induce the production of such enzymes. Various combinations of these ingredients in the “NO generating system” may be employed. The formulation and mode of administration will depend, of course, on the choice of the elements contained in the NO generating system.

NO donors are organic compounds that contain at least the N that is present in NO and behave as substrates (as opposed to inhibitors and as opposed to not being susceptible to relevant enzyme catalysis) of an NO generating enzyme. Organic NO donors include esters of simple organic alcohols or polyols with nitric acid or nitrous acid (e.g., isoamyl nitrite). Organic NO donors are exemplified by, but not limited to glycerol trinitrate (i.e., nitroglycerin), tetranitroerythritol, hexanitroinositol, tetranitropentaerythritol, propatyl nitrate, isosorbide 5-mononitrate (IS-5-MN), isosorbide dinitrate, isosorbide 2-mononitrate (IS-2-MN), isomannide 2-nitrate, 6-chloro-2-pyridylmethyl nitrate, and trinitrotriethanolamine, and their substituted derivatives, in particular the aminopropanol derivatives of 1,4:3,6-dianhydrohexitol nitrates, to the extent these compounds serve as substrates for NO generating enzymes or to the extent they donate N to form NO. Not all inorganic nitrates are NO donors since in some cases, inhibition of the relevant enzymes may result from contact with these nitrates. Useful organic NO donors can include, for instance, those disclosed in U.S. Pat. No. 5,591,758, incorporated herein by reference. These are of the formula:

R—CO—(A)ₙ—Y—B

wherein R represents, in particular, a sulfur-containing radical and a sulfur-containing amino acid residue; A represents a CH₂ group or a substituted amino acid; n is 0 or an integer; Y is an O or NH and B is an alcohol coupled to NO₂. In one embodiment, the organic NO donor is glycerol trinitrate nitroglycerin.

Any suitable organic NO donor known in the art can be used in the disclosed methods. Such organic NO donors include pharmaceutical compositions commercially available, e.g., Minitran, NT-1, Niotrocor, Nitroderm, Nitrodisc, Nitro-dur, Nitro-Dur II, Nitrofilm, Nitrogard, Nitroglin, Nitropen, Tridil, Isordil, Sorbitrate, Sorbitrate SA, Iso-Bid, Ismo, Nitrong, Nitro-Bid IV, Transderm-Nitro, and Nitrol.

Any suitable form of L-arginine can be used in or as the NO generating system. Such salts include 2,4-bisglyco-deuteroporphyrin L-arginate, 2,4-sulfonedeuteroporphyrin L-arginate, heme-L-arginate, arginine glutamate, arginine butyrate, L-arginine hydrochloride and the like. In one embodiment, the L-arginine is L-arginine hydrochloride. Additional suitable anions for such a salt of L-arginine include bromide, fluoride, iodide, borate, hypobromite, hypochlorite, nitrite, nitrate, hyponitrite, sulfate, disulfate, sulfite, sulfonate, phosphate, diphosphate, phosphite, phosphonate, diphosphonate, perchlorate, perchlorite, oxalate, malonate, succinate, lactate, carbonate, bicarbonate, acetate, benzoate, citrate, tosylate, permanganate, manganate, propanolate, propanoate, ethandioate, butanoate, propoxide, chromate, dichromate, selenate, orthosilicate, metasilicate, pertechnetate, technetate, dimethanolate, dimethoxide, thiocyanate, cyanate, isocyanate, 1,4-cyclohexanedithiolate, oxidobutanoate, 3-sulfidocyclobutane-1-sulfonate, 2-(2-carboxylatoethyl)-cyclohexanecarboxylate, 2-amino-4-(methylthio)-butanoate and the like. The suitable cation for most salts is hydrogen, however, other cations such as sodium, potassium and the like would be acceptable in the preparation of such a salt. It would be advantageous if the specific salt form selected allowed a pH close to neutral. Esters of L-arginine such as arginine ethyl ester or arginine butyl ester may also be used as well as other alkyl (ethyl, methyl, propyl, isopropyl, butyl, isobutyl, t-butyl) esters of L-arginine and salts thereof. Any derivative of L-arginine that acts as a precursor or donor of NO can be used in these methods, including any arginine derivative that is commercially available.

As mentioned above, the NO generating system may also comprise compounds that activate enzymes, either endogenous enzymes or enzymes supplied exogenously, that generate NO from NO donors. It is preferred to activate the eNOS forms of the enzyme and a number of compounds that upregulate eNOS expression and/or increase eNOS activity are known. These include cyclosporin A, FK506, felodipine, nicorandil, nifedipine, diltiazem, resveritrol, sapogrelate and quinapril. Among known activators of eNOS, interestingly, are the statins themselves. Mixtures of NO donors may have this effect as described in U.S. Pat. No. 5,543,430 which describes nitroglycerin as an eNOS agonist in combination with arginine. While cytokines are known to stimulate the production of inducible NOS, these stimulants must be provided cautiously as they are also associated with an inflammatory response. Cells modified to produce the relevant NO synthesizing enzymes may also be employed.

For clarity, it should be stated that by “NO generating system” is meant a component of the invention methods and compositions that results in the production of NO or the enhancement of such production. Thus, the “NO generating system” is not meant to include, necessarily, all components of the system, but simply whatever compound or compounds is employed in the compositions and methods to result in enhanced NO formation. Thus, the “NO generating system” may simply include an activator for eNOS or an activator of its production. It may simply include the NO donor or it may simply include the enzyme which converts the donor to NO and additional products or it may be a combination of these.

Statin-Like Compounds

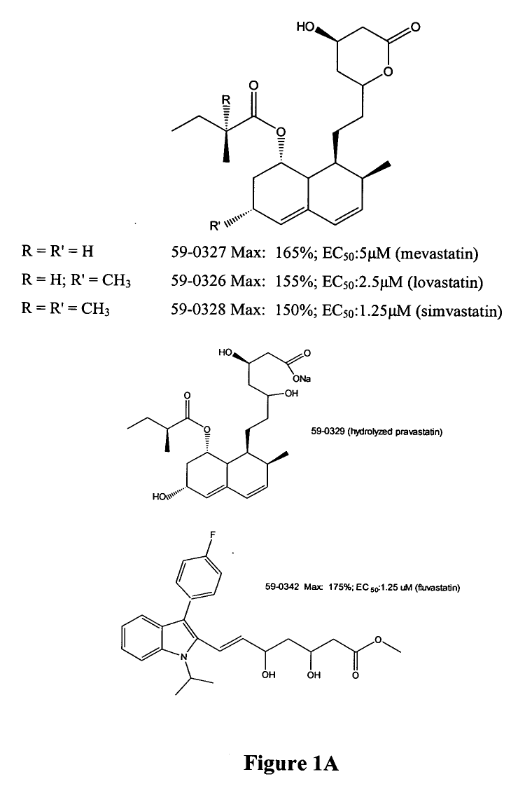

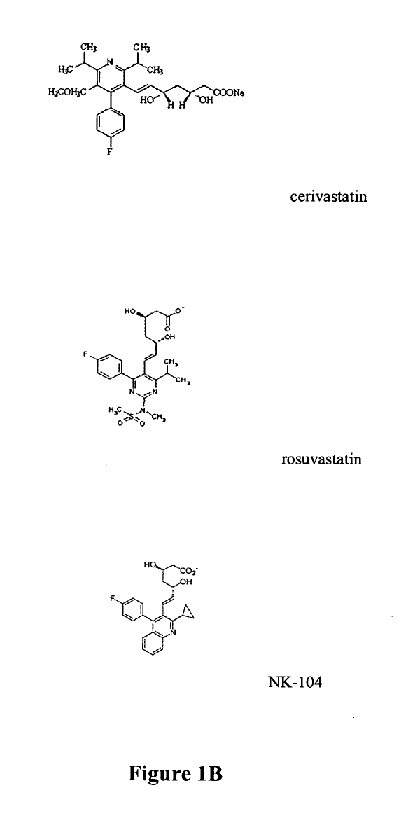

“Statins” per se are defined functionally as compounds that inhibit the enzyme 3-hydroxy-3-methylglutaryl coenzyme A (HMG-CoA) reductase, and thus typically used as drugs to inhibit cholesterol formation, or alternatively, they are defined structurally as those that contain the lactone or open lactone structure of formula (1) or (2). Known statins include cerivastatin, marketed as Baycol® by Bayer (See U.S. Pat. Nos. 5,006,530 and 5,177,080), lovastatin, marketed as Mevacor® by Merck (See U.S. Pat. No. 4,963,538), and simvastatin, marketed as Zocar®, pravastatin, marketed under as Pravachol®, atorvastatin, marketed as Lipotor® by Warner-Lambert (See U.S. Pat. No. 5,273,995), fluvastatin, marketed as Lescol® (See U.S. Pat. No. 4,739,073), and rosuvastatin, marketed as Crestor® (See, e.g., WO 02/41895). Another statin is NK-104 developed by NEGMA. See Akiba, T, et al., J Toxicol Sci (1998) 23V:713-720. The structures of these compounds are shown in FIG. 1. Additional compounds of similar structure or which inhibit enzymes of the isoprenoid/steroid pathway such as HMG-CoA reductase are defined as statin-like compounds. The ability of a compound to inhibit these enzymes can be determined by standard assays well known in the art, and as described below.

Although, in accordance with the description above, the statins might themselves be considered an “NO generating system”, because these compounds have a distinct status in the art they are placed in a separate category for purposes of the present invention.

The term “statin-like compounds” refers to compounds that have the property of inhibiting one or more enzymes that are participants in the isoprenoid/steroid synthetic pathway. Some of these compounds specifically inhibit HMG-CoA reductase; others inhibit different enzymes. For example, apamine and zaragozic acid inhibit enzymes in the pathway that are subsequent to the synthesis of the isoprenoids and are involved in the conversion of isoprenoid precursors to cholesterol. Bisphosphonates are also known to inhibit the isoprenoid/steroid pathway. Thus, included in the definition of “statin-like compounds” are any compounds that effect such inhibition, as well as compounds which contain the recognized statin type structure set forth as formulas (1) and (2) herein. A “statin-like compound” includes compounds that have either the functional definition provided above or the structural definition provided by formulas (1) or (2) or both.

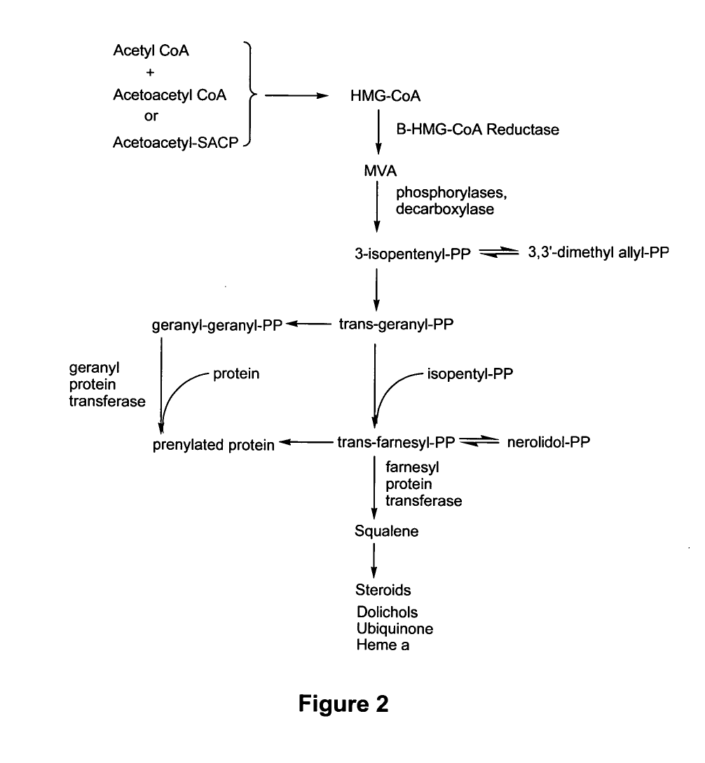

FIG. 2 is a diagram of the synthetic pathway which includes isoprenoid intermediates and ultimately results in the formation of steroids or the prenylation of proteins. As used herein, “isoprenoid/steroid pathway” refers to the conversions summarized in this figure, and “enzymes of the isoprenoid/steroid pathway” refers to any enzyme which catalyzes these conversions. More detailed descriptions of the individual conversions in the general outline shown in FIG. 2 will be found in standard texts on metabolism and biochemistry. The outline in FIG. 2 is intended as an overview only, and does not depict each and every conversion step. These are as have been elucidated over the past 40 or so years in the study of acetate metabolism. Compounds which inhibit these pathways, summarized by the diagram in FIG. 2, are defined as “statin-like compounds” and are useful in stimulating bone growth or inhibiting bone resorption or both. Compounds that inhibit the various steps in this pathway can easily be identified by assessing their ability to inhibit the particular enzymes that catalyze the relevant steps.

Such assays can be conducted by contacting said compound with an assay mixture for the activity of an enzyme in the isoprenoid pathway; determining the activity of the enzyme in the presence as compared to the absence of said compound; wherein a decrease of activity of said enzyme in the presence as opposed to the absence of said compound indicates that the compound is a statin that will be useful in treating bone disorders in vertebrates as described in U.S. Pat. Nos. 6,083,690, 6,022,887; 6,080,779 and 6,376,476. In addition, other compounds useful to enhance bone growth inhibit the production of isoprenoid/steroid pathway enzymes. These can be identified using assays employing a reporter gene under control of the expression control sequences for these enzymes as described in Yagi, Y., et al., Drug Development Research (1997) 40:41-47 and U.S. Pat. No. 6,083,690. Finally, since the enzymes involved in this pathway are known and often commercially available, simple in vitro assays for this inhibition activity are well within ordinary skill.

Any statin-like compound structurally defined as in formulas (1) and (2) is useful in the disclosed methods and compositions. One group of statin compounds useful in the disclosed methods and compositions has the formula:

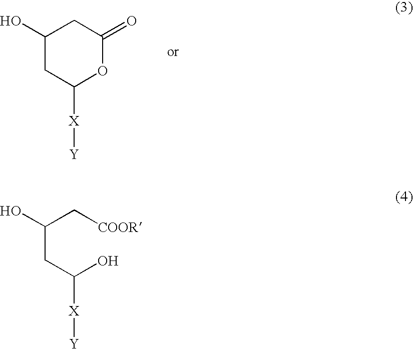

wherein X in each of formulas (3) and (4) represents substituted or unsubstituted alkylene (1-6C), alkenylene (2-6C), or alkynylene (2-6C);

Y comprises one or more carbocyclic or heterocyclic rings; when Y comprises two or more rings, they may optionally be fused; and

R′ represents a cation, H or a substituted or unsubstituted alkyl group of 1-6C. It is understood that if R′ represents a cation with multiple positive charges, the appropriate number of anions is coupled with it. Formulas (3) and (4) are, respectively, the unhydrolyzed and hydrolyzed forms of these statin compounds. Preferred substituents on X (or on R′ when R′ is alkyl) are hydroxy, alkoxy, phenyl, amino and alkyl- or dialkylamino.

The compounds statins typically contain at least one and generally several chiral centers. Compounds useful in the disclosed methods and composition include mixtures of the various stereoisomers and the stereoisomeric forms of the compounds individually. Preferred stereoisomers with respect to the compound of formula (1) are of the formula:

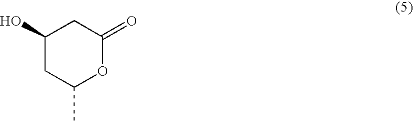

and the corresponding stereochemistry in the open chain (non-lactone or hydrolyzed) form of formula (2).

In one set of embodiments, X is unsubstituted; X may be, for example, —CH₂—; —CH₂CH₂—; —CH═CH—; or —C≡C—.

Typical embodiments of Y comprise ring systems such as naphthyl, polyhydro-naphthyl, monohydro- or dihydrophenyl, quinolyl, pyridyl, quinazolyl, pteridyl, pyrolyl, oxazoyl and the like and the reduced or partially reduced forms thereof.

Some embodiments of the substituent Y are those of the formula:

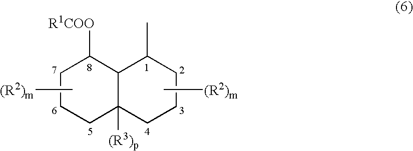

wherein the ring system may contain π-bonds;

wherein R¹ is substituted or unsubstituted alkyl;

each R² is independently a noninterfering substituent;

R³ is H, hydroxy, or alkoxy (1-6C);

each m is independently an integer of 0-6, wherein each R may reside in any of positions 2-7; and

p is 0 or 1, depending on the position of any 1-bonds.

Some embodiments of formula (6) are those of formulas (6a)-(6f) wherein the upper limit of n is adjusted according to the valence requirements appropriate for the particular ring system.

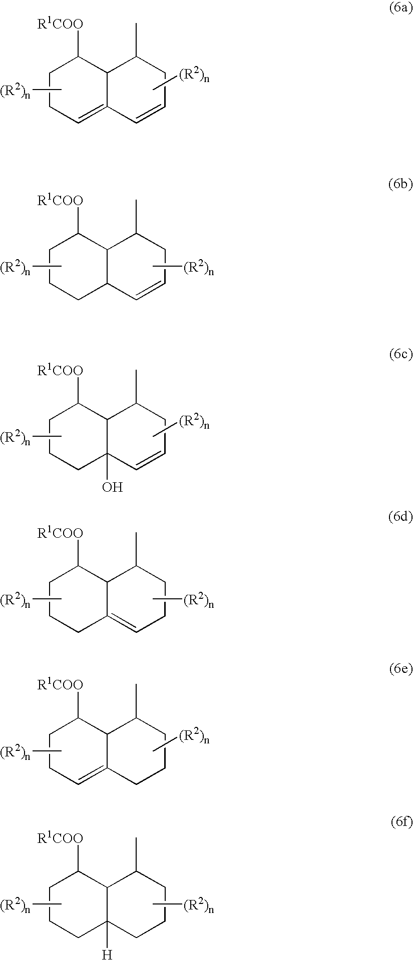

R¹ may be substituted alkyl, wherein the substituents may include hydroxy, alkoxy, alkylthiol, phenyl, phenylalkyl, and halo or unsubstituted alkyl is preferred. Particularly preferred embodiments of R¹ are alkyl of 1-6C, including propyl, sec-butyl, t-butyl, n-butyl, isobutyl, pentyl, isopentyl, 1-methylbutyl, and 2-methylbutyl. Particularly preferred are propyl and sec-butyl.

Preferred embodiments for R² include H, hydroxy, ═O, and substituted or unsubstituted lower alkyl (1-4C), in particular methyl, and hydroxymethyl. In the preferred embodiments, each n is independently 1 or 2 and preferred positions for substitution are positions 2 and 6 (see formula (6)). Particularly preferred embodiments of R² are OH, H, and lower alkyl, in particular CH₃.

Particularly preferred are embodiments wherein Y is 6(a) or 6(b), and especially embodiments having the substitution and chiral pattern indicated in formulas 6(g) and 6(h) below.

As indicated above, the compounds of the disclosed methods and compositions may be supplied as individual stereoisomers or as mixtures of stereoisomers. Preferred stereoisomers are those of the formulas (6g) and (6h) as typical and appropriate for those represented by the formulas (6a)-(6f).

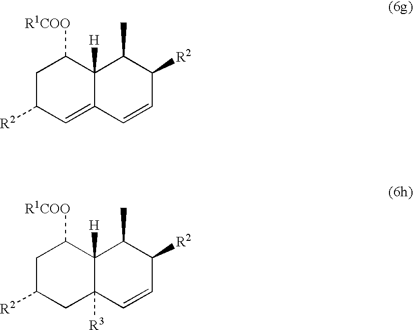

Particularly preferred are compounds with the stereochemistry of formulas (6g) and (6h) wherein the noted substituents are the sole substituents on the polyhydronaphthyl system optionally including additional substituents at position 5. Preferred embodiments include those wherein each of R² is independently OH, CH₂OH, methyl, or ═O. Preferred embodiments of R¹ in these preferred forms are propyl, sec-butyl, and 2-methyl-but-2-yl.

Additional preferred embodiments of Y are:

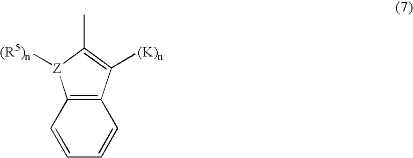

wherein Z is N and both n are 1, and each K comprises a substituted or unsubstituted aromatic or nonaromatic carbocyclic or heterocyclic ring system which may optionally be spaced from the linkage position shown in formula (7) by a linker of 1-2C, including —CHOH—, —CO—, and —CHNH₂—, for example. Aromatic ring systems are preferred. Particularly preferred are compounds of formula (7), either as shown or wherein Z is contained in a 6-membered, rather than a 5-membered aromatic ring. Thus, another group of preferred compounds of the invention is of formula (7) where Z is N and an additional substituent—=CR⁶—is inserted between Z and the bond directed to X, wherein R6 is linear, branched or cyclic alkyl. In a preferred embodiment, R⁶ is a cyclic alkyl substituent.

R⁵ is H or linear, branched, cyclic substituted or unsubstituted alkyl, wherein substituents are preferably hydroxy, alkoxy, phenyl, amino and alkyl- or dialkylamino. Preferably, when R⁵ is alkyl, it is unsubstituted.

The substituents on the aromatic ring systems or nonaromatic ring systems of the invention including those designated by K can be any noninterfering substituents. Generally, the non-interfering substituents can be of wide variety. Among substituents that do not interfere with the beneficial effect of the compounds of the invention on bone formation in treated subjects include alkyl (1-6C, preferably lower alkyl 1-4C), including straight, branched or cyclic forms thereof, alkenyl (2-6C, preferably 2-4C), alkynyl (2-6C, preferably 2-4C), all of which can be straight or branched chains and may contain further substituents; halogens, including F, Cl, Br and I; silyloxy, OR, SR, NR₂, OOCR, COOR, NCOR, NCOOR, and benzoyl, CF₃, OCF₃, SCF₃, N(CF₃)₂, CN, SO, SO₂R and SO₃R wherein R is alkyl (1-6C) or is H. Where two substituents are in adjacent positions in the aromatic or nonaromatic system, they may form a ring. Further, rings not fused to the aromatic or nonaromatic system K may be included as substituents. These rings may be aromatic and may be substituted or unsubstituted.

Preferred non-interfering substituents include hydrocarbyl groups of 1-6C, including saturated and unsaturated, linear or branched hydrocarbyl as well as hydrocarbyl groups containing ring systems; halo groups, alkoxy, hydroxy, CN, CF₃, and COOR, amino, monoalkyl- and dialkylamino where the alkyl groups are 1-6C. Particularly preferred are substituted or unsubstituted aromatic rings.

Although the number of substituents on a ring symbolized by K may typically be 0-4 or 0-5 depending on the available positions, preferred embodiments include those wherein the number on a single ring is 0, 1 or 2, preferably 0 or 1. However, an exception is that of formula (8), where it is preferred that the aromatic carbocyclic or heterocyclic ring system be multiply substituted. In particular, it is preferred that the substituents on K in formula (8) themselves contain aromatic rings. Particularly preferred are substituents that contain phenyl rings.

Particularly preferred are the embodiments of formula (7) or the ring expanded form thereof wherein K represents optionally substituted phenyl. Particularly preferred are compounds wherein R⁵ is isopropyl and K is para fluorophenyl forms.

The compounds useful in the disclosed methods and compositions can be synthesized by art-known methods as they resemble a class of compounds known in the art to behave as antihypercholesterolemic agents. Typical among these is lovastatin, marketed by Merck as Mevacor®. The synthesis of lovastatin and various analogs thereof is set forth in U.S. Pat. No. 4,963,538. In addition, methods for synthesis of lovastatin and analogous compounds such as compactin (mevastatin), simvastatin, and pravastatin are set forth in U.S. Pat. Nos. 5,059,696; 4,873,345; 4,866,186; 4,894,466; 4,894,465; 4,855,456; and 5,393,893. Certain of these compounds are also produced by microorganisms as described in U.S. Pat. Nos. 5,362,638; 5,409,820; 4,965,200; and 5,409,820. Compounds described as end-products in these documents are useful in the methods of the invention.

Additional analogs, including those containing aromatic embodiments of Y, are described in U.S. Pat. No. 5,316,765. For example, the preparation of fluvastatin is described in PCT Application WO 84/02131. Other compounds are described in, for example, Roth, B. D., et al., J Med Chem (1991) 34:357-366; Krause, R., et al., J Drug Develop. (1990) 3(Suppl. 1):255-257v; Karanewsky, D. S., et al., J Med Chem (1990) 33:2952-2956.

Convenient for use in the method of the invention are hydrolyzed or unhydrolyzed forms of lovastatin, mevastatin, simvastatin, fluvastatin, pravastatin, cerivastatin, rosuvastatin, NK-104, and atorvastatin. Typical forms of these statins are shown in FIGS. 1A and 1B.

Phosphodiesterase Inhibitors

A large number of PDE inhibitors is known in the art. Some of these are non-specific with regard to the various families of phosphodiesterases; others are specific to particular PDE's. The ability of an agent to inhibit a phosphodiesterase is readily assayed using standard enzymological methods; any candidate compound considered a potential PDE inhibitor can be verified to have this activity using these assays.

Included among the PDE inhibitors useful in the invention are those that are non-specific for PDE's such as caffeine, theophylline, pentoxifylline and 3-isobutyl-1-methylxanthine. Also useful in the methods of the invention are PDE-4 inhibitors such as rolipram and XT-44. Other inhibitors are described in Wakabayashi, S., J. Bone Min. Res. (2000) 15:Suppl. 1 Abstract M198 and U.S. Pat. No. 6,010,711 cited previously. Other inhibitors useful are dipyridamole, MY-5445, sildenafil and Zaprinast™. Any compound shown to be a successful PDE inhibitor is useful in the methods of the present invention.

In addition, PDE inhibitors may include antibodies that bind specifically to PDE, including fragments of such antibodies, as well as aptamers and peptidomimetics, and includes protocols and agents which inhibit the synthesis of phosphodiesterases, such as inhibitory RNA, antisense constructs, and the like.

In Vitro and In Vivo Efficacy Assays

The methods of the invention can be shown to enhance bone growth in suitable assays as described below.

In Vitro Neonatal Mouse Calvaria Assay: An assay for bone resorption or bone formation is similar to that described by Gowen, M., & Mundy, G., J Immunol (1986) 136:2478-2482. Briefly, four days after birth, the front and parietal bones of ICR Swiss white mouse pups are removed by microdissection and split along the sagittal suture. In an assay for resorption, the bones are incubated in BGJb medium (Irvine Scientific, Santa Ana, Calif.) plus 0.02% (or lower concentration) β-methylcyclodextrin, wherein the medium also contains test or control substances. The medium used when the assay is conducted to assess bone formation is Fitton and Jackson Modified BGJ Medium (Sigma) supplemented with 6 μg/ml insulin, 6 μg/ml transferrin, 6 ng/ml selenous acid, calcium and phosphate concentrations of 1.25 and 3.0 mM, respectively, and ascorbic acid to a concentration of 100 μg/ml is added every two days. The incubation is conducted at 37° C. in a humidified atmosphere of 5% CO₂ and 95% air for 96 hours.

Following this, the bones are removed from the incubation media and fixed in 10% buffered formalin for 24-48 hours, decalcified in 14% EDTA for 1 week, processed through graded alcohols; and embedded in paraffin wax. Three μm sections of the calvaria are prepared. Representative sections are selected for histomorphometric assessment of bone formation or bone resorption. Bone changes are measured on sections cut 200 μm apart. Osteoblasts and osteoclasts are identified by their distinctive morphology.

In Vivo Assay of Effects on Murine Calvarial Bone Growth: Male ICR Swiss white mice, aged 4-6 weeks and weighing 13-26 gm, are employed, using 4-5 mice per group. The calvarial bone growth assay is performed as described in PCT application WO 95/24211, incorporated by reference. Briefly, the test protocol or appropriate control administered into the subcutaneous tissue over the right calvaria of normal mice. Typically, the control is the vehicle in which a compound to be tested was solubilized, and is PBS containing 5% DMSO or is PBS containing Tween (2 μl/10 ml). The animals are sacrificed on day 14 and bone growth measured by histomorphometry. Bone samples for quantitation are cleaned from adjacent tissues and fixed in 10% buffered formalin for 24-48 hours, decalcified in 14% EDTA for 1-3 weeks, processed through graded alcohols; and embedded in paraffin wax. Three to five μm sections of the calvaria are prepared, and representative sections are selected for histomorphometric assessment of the effects on bone formation and bone resorption. Sections are measured by using a camera Lucida attachment to trace directly the microscopic image onto a digitizing plate. Bone changes are measured on sections cut 200 μm apart, over 4 adjacent 1×1 mm fields on both the injected and non-injected sides of the calvaria. New bone is identified by its characteristic woven structure, and osteoclasts and osteoblasts are identified by their distinctive morphology. Histomorphometry software (OsteoMeasure, Osteometrix, Inc., Atlanta) is used to process digitizer input to determine cell counts and measure areas or perimeters.

Additional In Vivo Assays: Lead compounds and protocols can be further tested in intact animals using an in vivo, dosing assay. Prototypical dosing may be accomplished by subcutaneous, intraperitoneal or oral administration, and may be performed by injection, sustained release or other delivery techniques. The time period for administration of test compound may vary (for instance, 28 days as well as 35 days may be appropriate). An exemplary, in vivo oral or subcutaneous dosing assay may be conducted as follows:

In a typical study, 70 three-month-old female Sprague-Dawley rats are weight-matched and divided into seven groups, with ten animals in each group. This includes a baseline control group of animals sacrificed at the initiation of the study; a control group administered vehicle only; a PBS-treated control group; and a positive control group administered a compound (non-protein or protein) known to promote bone growth. Three dosage levels of the compound or protocol to be tested are administered to the remaining three groups.

Briefly, test compound(s) or protocol(s), positive control compound, PBS, or vehicle alone is administered subcutaneously once per day for 35 days. All animals are injected with calcein nine days and two days before sacrifice (two injections of calcein administered each designated day). Weekly body weights are determined. At the end of the 35-day cycle, the animals are weighed and bled by orbital or cardiac puncture. Serum calcium, phosphate, osteocalcin, and CBCs are determined. Both leg bones (femur and tibia) and lumbar vertebrae are removed, cleaned of adhering soft tissue, and stored in 70% ethanol for evaluation, as performed by peripheral quantitative computed tomography (pQCT; Ferretti, J., Bone (1995) 17:353S-64S), dual energy X-ray absorptiometry (DEXA; Laval-Jeantet, A., et al., Calcif Tissue Intl (1995) 56:14-18; Casez, J., et al., Bone and Mineral (1994) 26:61-68) and/or histomorphometry. The effect of test compounds or protocols on bone remodeling can thus be evaluated.

Protocols can also be tested in acute ovariectomized animals (prevention model) using an in vivo dosing assay. Such assays may also include an estrogen-treated group as a control. An exemplary subcutaneous dosing assay is performed as follows:

In a typical study, 80 three-month-old female Sprague-Dawley rats are weight-matched and divided into eight groups, with ten animals in each group. This includes a baseline control group of animals sacrificed at the initiation of the study; three control groups (sham ovariectomized (sham OVX)+vehicle only; ovariectomized (OVX)+vehicle only; PBS-treated OVX); and a control OVX group that is administered a compound known to promote bone growth. Three dosage levels of the compound to be tested are administered to the remaining three groups of OVX animals.

Since ovariectomy (OVX) induces hyperphagia, all OVX animals are pair-fed with sham OVX animals throughout the 35 day study. Briefly, test compound, positive control compound, PBS, or vehicle alone is administered orally or subcutaneously once per day for 35 days. Alternatively, test compound can be formulated in implantable pellets that are implanted for 35 days, or may be administered orally, such as by gastric gavage. All animals, including sham OVX/vehicle and OVX/vehicle groups, are injected intraperitoneally with calcein nine days and two days before sacrifice (two injections of calcein administered each designated day, to ensure proper labeling of newly formed bone). Weekly body weights are determined. At the end of the 35-day cycle, the animals' blood and tissues are processed as described above.

Protocols may also be tested in chronic OVX animals (treatment model). An exemplary protocol for treatment of established bone loss in ovariectomized animals that can be used to assess efficacy of anabolic agents may be performed as follows. Briefly, 80 to 100 six month old female, Sprague-Dawley rats are subjected to sham surgery (sham OVX) or ovariectomy (OVX) at time 0, and 10 rats are sacrificed to serve as baseline controls. Body weights are recorded weekly during the experiment. After approximately 6 weeks (42 days) or more of bone depletion, 10 sham OVX and 10 OVX rats are randomly selected for sacrifice as depletion period controls. Of the remaining animals, 10 sham OVX and 10 OVX rats are used as placebo-treated controls. The remaining OVX animals are treated with 3 to 5 doses of test drug for a period of 5 weeks (35 days). As a positive control, a group of OVX rats can be treated with an agent such as PTH, a known anabolic agent in this model (Kimmel, et al., Endocrinology (1993) 132:1577-1584). To determine effects on bone formation, the following procedure can be followed. The femurs, tibiae and lumbar vertebrae 1 to 4 are excised and collected. The proximal left and right tibiae are used for pQCT measurements, cancellous bone mineral density (BMD) (gravimetric determination), and histology, while the midshaft of each tibiae is subjected to cortical BMD or histology. The femurs are prepared for pQCT scanning of the midshaft prior to biomechanical testing. With respect to lumbar vertebrae (LV), LV2 are processed for BMD (PQCT may also be performed); LV3 are prepared for undecalcified bone histology; and LV4 are processed for mechanical testing.

Administration

The methods provided herein can employ any suitable composition(s) administered in any suitable manner to achieve an anabolic effect on bone. Bone or cartilage deficit or defect can be treated in vertebrate subjects by the disclosed methods using compositions comprising compounds that exhibit the required structural and functional characteristics. The compositions may include one or more active compounds. Clearly, the preferred mode of administration will depend on the selection of the particular statins, PDE inhibitor and, more importantly, on the nature of the NO-generating system employed.

Statins and most PDE inhibitors are individual compounds, so that these compounds or mixtures of statins and/or PDE inhibitors are administered in a manner conventional for “small molecule” administration. Such methods are described in detail below. Similarly, if the NO-generating system simply consists of one or more of NO donors, these compounds, also as “small molecules” can be administered in a conventional manner. The compounds may also be provided as conjugates, for example with agents that affect half-life, such as PEG, and/or with a labeling components such as a nuclide or dye and/or with a targeting agent, such as a ligand or antibody.

The (NO donor and the statin) or (NO donor and PDE inhibitor) or (statin and PDE inhibitor) or all three components may be administered simultaneously, in the same composition or in separate compositions, or may be administered sequentially. Appropriate protocols are determinable using the assay systems set forth above as guides and using routine optimization procedures employed in medical and veterinary treatments generally. If the nitrogen-generating system includes an enzyme that catalyzes the production of NO, alternative methods of administering this enzyme may be desirable. Typically, enzymatically active proteins are administered by injection, most conveniently intravenously. The administration of the enzyme would in most cases be conducted separately from the administration of the statin or PDE inhibitor, although a combined composition might be introduced intravenously. Both an NO-generating enzyme and its NO donor substrate may be used in the same protocol or only one or the other employed.

The NO-generating enzyme may be recombinantly produced by introducing naked DNA or a viral vector for generating an expression system for this enzyme. These may be administered directly to the subject or the subject's cells may be altered ex vivo to produce this enzyme and reinfused into the subject. The cells producing the enzyme may, if desired, be administered in combination with the substrate NO donor as the NO-generating system component of the invention method.

In still another alternative or additive feature of the NO-generating system, a compound that stimulates the production of the NO-generating enzyme may be employed in the invention method. Such compounds may, for example, include cytokines that are known to induce the inducible form of NOS. Compounds that activate the enzyme may also be included.

Similarly, alternative methods may be desirable for administering antibodies or fragments thereof that are inhibitors of phosphodiesterases and alternative methods are useful as well for administering antisense or inhibitory nucleic acids which interfere with the synthesis of the phosphodiesterases.

In all cases, the various components of the bone-enhancing protocol may be introduced simultaneously or sequentially.

In general, compounds may be administered systemically or locally. For systemic use, the compositions herein are formulated for parenteral (e.g., buccal, intravenous, subcutaneous, intramuscular, intraperitoneal, intranasal, sublingual, or transdermal) or enteral (e.g., oral or rectal) delivery according to conventional methods. Intravenous administration can be by a series of injections or by continuous infusion over an extended period. Administration by injection or other routes of discretely spaced administration can be performed at intervals ranging from weekly to once to three times daily. Administration routes and dosing may be the same or different for the different compositions used in the disclosed methods. In one embodiment, a pharmaceutical composition comprising a statin and an organic nitrate is administered orally as a single composition (e.g., as a tablet). Alternatively, the compositions disclosed herein may be administered in a cyclical manner (administration of a component compound; followed by no administration; followed by administration of component, and the like). Treatment will continue until the desired outcome is achieved.

In general, pharmaceutical formulations of compositions will include at least one compound useful in the present methods in combination with a pharmaceutically acceptable vehicle, such as saline, buffered saline, 5% dextrose in water, borate-buffered saline containing trace metals or the like. Formulations may further include one or more excipients, preservatives, solubilizers, buffering agents, albumin to prevent protein loss on vial surfaces, lubricants, fillers, stabilizers, etc. Methods of formulation are well known in the art and are disclosed, for example, in Remington's Pharmaceutical Sciences, latest edition, Mack Publishing Co., Easton Pa. In one embodiment, the pharmaceutical composition include a statin compound with an organic nitrate. In a specific embodiment, the pharmaceutical composition comprises atorvastatin and glyceryl trinitrate.

Pharmaceutical compositions for use within the disclosed methods can be in the form of sterile, non-pyrogenic liquid solutions or suspensions, coated capsules, suppositories, lyophilized powders, transdermal patches or other forms known in the art. Local administration may be by injection at the site of injury or defect, or by insertion or attachment of a solid carrier at the site, or by direct, topical application such as of a viscous liquid, or the like. For local administration, the delivery vehicle may provide a matrix for the growing bone or cartilage, and may be a vehicle that can be absorbed by the subject without adverse effects.

Delivery of compositions herein to specific sites may be enhanced by the use of controlled-release compositions, such as those described in PCT application WO 93/20859. Films of this type are particularly useful as coatings for prosthetic devices and surgical implants. The films may, for example, be wrapped around the outer surfaces of surgical screws, rods, pins, plates and the like. Implantable devices of this type are routinely used in orthopedic surgery. The films can also be used to coat bone filling materials, such as hydroxyapatite blocks, demineralized bone matrix plugs, collagen matrices and the like. In general, a film or device as described herein is applied to the bone at the fracture site. Application is generally by implantation into the bone or attachment to the surface using standard surgical procedures.

In addition to the copolymers and carriers noted above, the biodegradable films and matrices may include other active or inert components. Of particular interest are those agents that promote tissue growth or infiltration, such as growth factors. Exemplary growth factors for this purpose include epidermal growth factor (EGF), fibroblast growth factor (FGF), platelet-derived growth factor (PDGF), transforming growth factors (TGFs), parathyroid hormone (PTH), leukemia inhibitory factor (LIF), insulin-like growth factors (IGFs) and the like. Agents that promote bone growth, such as bone morphogenetic proteins (U.S. Pat. No. 4,761,471; PCT Publication WO 90/11366), osteogenin (Sampath, et al., Proc. Natl. Acad. Sci. USA (1987) 84:7109-7113) and NaF (Tencer, et al., J. Biomed. Mat. Res. (1989) 23:571-589) are also contemplated. Biodegradable films or matrices include calcium sulfate, tricalcium phosphate, hydroxyapatite, polylactic acid, polyanhydrides, bone or dermal collagen, pure proteins, extracellular matrix components and the like and combinations thereof. Such biodegradable materials may be used in combination with non-biodegradable materials, to provide desired mechanical, cosmetic or tissue or matrix interface properties.

Alternative methods for delivery of compositions of the present invention include use of ALZET osmotic minipumps (Alza Corp., Palo Alto, Calif.); sustained release matrix materials such as those disclosed in Wang, et al., (PCT Publication WO 90/11366); electrically charged dextran beads, as disclosed in Bao, et al., (PCT Publication WO 92/03125); collagen-based delivery systems, for example, as disclosed in Ksander, et al., Ann. Surg. (1990) 211(3):288-294; methylcellulose gel systems, as disclosed in Beck, et al., J Bone Min. Res. (1991) 6(11):1257-1265; alginate-based systems, as disclosed in Edelman, et al., Biomaterials (1991) 12:619-626 degradable polymeric systems, such as those disclosed in WO 02/082973, or vinyl pyrrolidine polymers as disclosed in 4,533,540, and the like. Other methods well known in the art for sustained local delivery in bone include porous coated metal prostheses that can be impregnated and solid plastic rods with therapeutic compositions incorporated within them.

The compositions useful in the present methods may also be used in conjunction with agents that inhibit bone resorption. Antiresorptive agents, such as estrogen, bisphosphonates and calcitonin, are preferred for this purpose. More specifically, the compositions disclosed herein may be administered for a period of time (for instance, months to years) sufficient to obtain correction of a bone deficit or disorder. Once the bone disorder has been corrected, the vertebrate can be administered an anti-resorptive compound to maintain the corrected bone condition. Alternatively, the compositions disclosed herein may be administered with an anti-resorptive compound in a cyclical manner (administration of disclosed compound, followed by anti-resorptive, followed by disclosed compound, and the like).

In additional formulations, conventional preparations such as those described below may be used.

Aqueous suspensions may contain the active ingredient in admixture with pharmacologically acceptable excipients, comprising suspending agents, such as methyl cellulose; and wetting agents, such as lecithin, lysolecithin or long-chain fatty alcohols. The said aqueous suspensions may also contain preservatives, coloring agents, flavoring agents, sweetening agents and the like in accordance with industry standards.

Preparations for topical and local application comprise aerosol sprays, lotions, gels and ointments in pharmaceutically appropriate vehicles which may comprise lower aliphatic alcohols, polyglycols such as glycerol, polyethylene glycol, esters of fatty acids, oils and fats, and silicones. The preparations may further comprise antioxidants, such as ascorbic acid or tocopherol, and preservatives, such as p-hydroxybenzoic acid esters.

Parenteral preparations comprise particularly sterile or sterilized products. Injectable compositions may be provided containing the active compound and any of the well known injectable carriers. These may contain salts for regulating the osmotic pressure.

Preparation for oral administration include a pharmaceutically acceptable vehicle such as an inert diluent or an assimilable edible carrier. They may be enclosed in hard or soft shell gelatin capsules, compressed into tablets, or incorporated directly with the food of the patient's diet. For oral therapeutic administration, the composition may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

If desired, the compositions can be incorporated into liposomes by any of the reported methods of preparing liposomes for use in treating various pathogenic conditions. The present compositions may utilize the compounds noted above incorporated in liposomes in order to direct these compounds to macrophages, monocytes, as well as other cells and tissues and organs which take up the liposomal composition. The liposome-incorporated compositions in the present methods can be utilized by parenteral administration, to allow for the efficacious use of lower doses of the compounds. Ligands may also be incorporated to further focus the specificity of the liposomes.

Suitable conventional methods of liposome preparation include, but are not limited to, those disclosed by Bangham, A. D., et al., J Mol Biol (1965) 23:238-252, Olson, F., et al., Biochim Biophys Acta (1979) 557:9-23, Szoka, F., et al., Proc Natl Acad Sci USA (1978) 75:4194-4198, Kim, S., et al., Biochim Biophys Acta (1983)728:339-348, and Mayer, et al., Biochim Biophys Acta (1986) 858:161-168.

The liposomes may be made from the present compounds in combination with any of the conventional synthetic or natural phospholipid liposome materials including phospholipids from natural sources such as egg, plant or animal sources such as phosphatidylcholine, phosphatidylethanolamine, phosphatidylglycerol, sphingomyelin, phosphatidylserine, or phosphatidylinositol and the like. Synthetic phospholipids that may also be used, include, but are not limited to: dimyristoylphosphatidylcholine, dioleoylphosphatidylcholine, dipalmitoylphosphatidylcholine and distearoylphosphatidycholine, and the corresponding synthetic phosphatidylethanolamines and phosphatidylglycerols. Cholesterol or other sterols, cholesterol hemisuccinate, glycolipids, cerebrosides, fatty acids, gangliosides, sphingolipids, 1,2-bis(oleoyloxy)-3-(trimethyl ammonio) propane (DOTAP), N-[1-(2,3-dioleoyl) propyl-N,N,N-trimethylammonium chloride (DOTMA), and other cationic lipids may be incorporated into the liposomes, as is known to those skilled in the art. The relative amounts of phospholipid and additives used in the liposomes may be varied if desired. The preferred ranges are from about 60 to 90 mole percent of the phospholipid; cholesterol, cholesterol hemisuccinate, fatty acids or cationic lipids may be used in amounts ranging from 0 to 50 mole percent. The amounts of the present compounds incorporated into the lipid layer of liposomes can be varied with the concentration of the lipids ranging from about 0.01 to about 50 mole percent.

The liposomes with the above formulations may be made still more specific for their intended targets with the incorporation of monoclonal antibodies or other ligands specific for a target. For example, monoclonal antibodies to the BMP receptor may be incorporated into the liposome by linkage to phosphatidylethanolamine (PE) incorporated into the liposome by the method of Leserman, L., et al., Nature (1980) 288:602-604.

The dosage of the compounds of the invention will vary according to the extent and severity of the need for treatment, the activity of the administered composition, the general health of the subject, and other considerations well known to the skilled artisan. Generally, they can be administered to a typical human on a daily basis as an oral dose of about 0.1 mg/kg-1000 mg/kg, and more preferably from about 1 mg/kg to about 200 mg/kg. The parenteral dose will appropriately be 20-100% of the oral dose. While oral administration may be preferable in most instances (for reasons of ease, patient acceptability, and the like), alternative methods of administration may be appropriate for selected compounds and selected defects or diseases. In some embodiments, one composition may be administered subcutaneously, while the statin-type composition is administered orally.

In addition to the statin-like compounds, nitric oxide generating systems, and PDE inhibitors provided herein, the compositions may also include other agents, including those which stimulate bone formation and/or inhibit bone resorption such as estrogens or their analogs and/or compounds of the formula Ar-L-Ar wherein Ar represents an aryl substituent and L represents a linker, such as those disclosed in PCT publications WO 98/17267 published 30 Apr. 1998, WO 97/15308 published 1 May 1997 and WO 97/48694 published 24 Dec. 1997. Any suitable bone enhancer or bone resorption inhibitor can be used in a combination therapy as the additional agent. Exemplary compounds that can be used in the combination therapy include steroids, bone growth stimulating compounds, bone morphogenetic factors, anti-resorptive agents, osteogenic factors, cartilage-derived morphogenetic proteins, growth hormones, estrogens, bisphosphonates, differentiating factors, compounds that inhibit activity of NF-κB, compounds that inhibit production of NF-κB, compounds that inhibit activity of proteasomal activity and compounds that inhibits production of a proteasome protein. Other useful compounds are disclosed in PCT publications PCT/US 00/41360, filed 20 Oct. 2000 and WO 00/02548. Also useful are microtubule formation inhibitors, such as 3-(1-Anilinoethylidene)-5-benzylpyrrolidine-2,4-dione (TN-16); N-(5,6,7,9-Tetrahydro-1,2,3,10-tetra-methoxy-9-oxobenzo [a]heptalen-7-yl) acetamide (Colchicine); Methyl-[5-(2-thienylcarbonyl)-1H-benzimidazole-2-yl]-carbamate (Nocodazole); and 2-Methoxy-estradiol (2-ME), and analogs, such as paclitaxel, docetaxel, taxane, other benzimidazole carbamates, ansamitocin, and the like.

The following examples are intended to illustrate but not to limit the invention.

### EXAMPLE 1

### Stimulation of Bone Formation by L-Arginine and Statins

Selected compounds and appropriate controls were assayed in vitro (ex vivo) for bone formation activity (described above in “In Vitro Neonatal Mouse Calvaria Assay”). Histomorphometrical assessments of ex vivo calvaria were carried out using an OsteoMetrics bone morphometry measurement program, according to the manufacturer's instructions. Measurements were determined using either a 10- or 20-fold objective with a standard point counting eyepiece graticule.

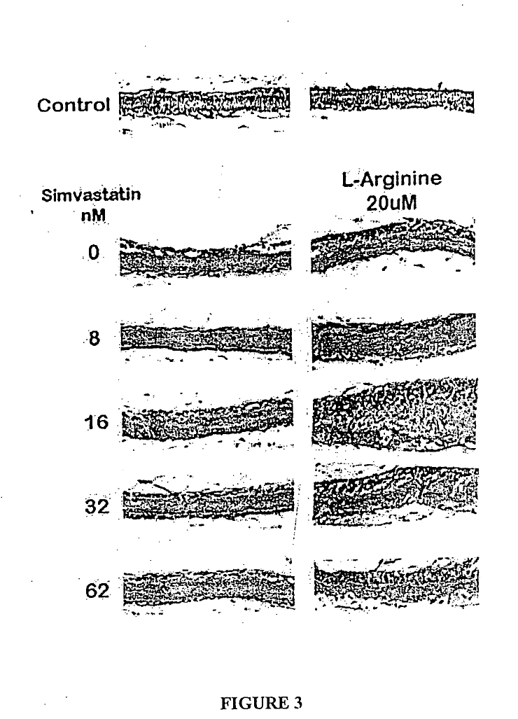

FIG. 3 shows the results of the assay using simvastatin alone (left panel), L-arginine alone (second panel from top right), and in combination at various concentrations (remaining panels). Control panels for each are shown at the top of FIG. 3. These data show that simvastatin alone induces bone formation in a dose dependent manner as does 20 μM L-arginine. However, the combination of simvastatin and L-arginine show a synergistic increase in bone formation when administered together.

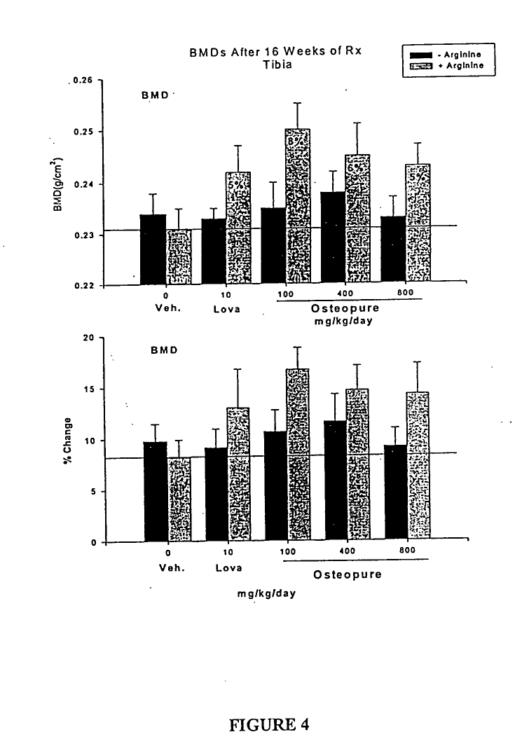

FIG. 4 shows the effects of lovastatin and arginine in stimulating bone growth. In this experiment, experimental groups of three month old S-D rats were used. Food was removed from the cages 6 hours prior to dosing. The animals were then treated orally by gavage once a day with vehicle (0.5% methylcellulose), lovastatin, or OsteoPure™ (lovastatin-containing reduced rice extract). OsteoPure™ was administered at 100, 400, or 800 mg/kg/day five days a week for 16 weeks. The statin equivalence in OsteoPure™ is 100 mg/kg/day=0.72 mg/day; 400 mg/kg/day=2.9 mg/day; and 800 mg/kg/day=5.8 mg/day. Rats were weighed weekly. Bone mineral density (BMD) were analyzed every four weeks. In the upper panel, the bone mineral densities (BMDs) are shown. In the lower panel, the % change in BMDs of the experimental over the 16 weeks is shown. The data in the upper panel shows a 5% increase in BMDs with 10 mg/kg/day of lovastatin and L-arginine over the BMD with lovastatin alone. Co-administration of OsteoPure™ with L-arginine results in an increase in BMDs at every dose. This trend is confirmed in the long term analysis of percent change in BMDs shown in the lower panel. Lovastatin and OsteoPure™ show a greater increase in BMDs when co-administered with L-arginine relative to the increases in BMDs seen with lovastatin or OsteoPure™ alone.

Modifications may be made to the foregoing without departing from the basic aspects of the invention. Although the invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow.

Citation of the above publications or documents is not intended as an admission that any of the foregoing is pertinent prior art, nor does it constitute any admission as to the contents or date of these publications or documents. U.S. patents and other publications referenced herein are hereby incorporated by reference.

1. A method to enhance bone formation in a vertebrate subject which method comprises administering to a vertebrate subject in need of such enhancement an effective amount of any two of the following components
a) at least one nitric oxide (NO) generating system;
b) at least one statin-like compound; and
c) at least one phosphodiesterase (PDE) inhibitor.

2. The method of claim 1, wherein the statin-like compound is of the formula: 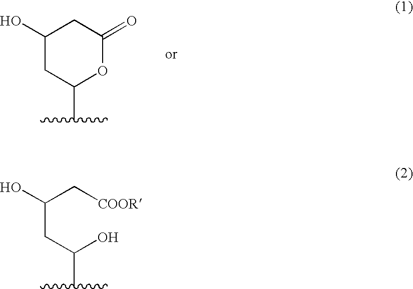
wherein each of formulas (1) and (2) is coupled through the indicated bond to an organic moiety of up to 40C.

3. The method of claim 2, wherein each of formulas (1) and (2) is coupled to —X-Y
wherein X represents substituted or unsubstituted alkylene (1-6C), alkenylene (2-6C), or alkynylene (2-6C); and
Y comprises one or more carbocyclic and/or heterocyclic rings.

4. The method of claim 3, wherein Y is of the formula 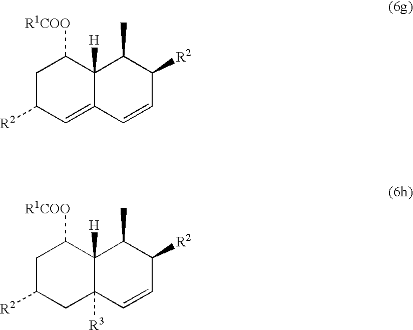
or a stereoisomer or mixture of stereoisomers thereof,
wherein R¹ is substituted or unsubstituted alkyl;
each R² is independently H, hydroxy, alkoxy (1-6C) or lower alkyl (1-4C);
R³ is H, hydroxy, or alkoxy (1-6C); or
Y is of the formula 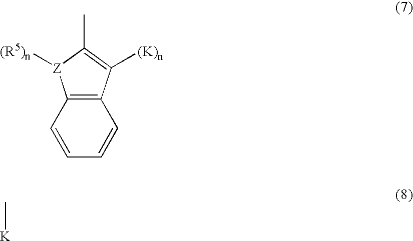
wherein each n is 1,
Z is N,
K comprises a substituted or unsubstituted aromatic carbocyclic or heterocyclic ring system which may optionally be spaced from the linkage position shown in formula (7) by a linker of 1-2C, or in formula (7), Z may be spaced from the carbon bonded to X by ═CR⁶-wherein R⁶ is H or linear, branded or cyclic alkyl (1-6C),
R⁵ is H or linear, branched or cyclic alkyl, and
R′ represents a cation, H or a substituted or unsubstituted alkyl group of 1-6C.

5. The method of claim 3 wherein X is selected from the group consisting of CH₂, —CH₂CH₂—; —CH═CH—; and —C≡C—.

6. The method of claim 5 wherein Y is of the formula (6g) or a stereoisomer or mixture of stereoisomers thereof.

7. The method of claim 6 wherein R¹ alkyl 4-5C.

8. The method of claim 6 wherein each R² is independently H, methyl or hydroxy.

9. The method of claim 5 wherein Y is of formula (7) as shown.

10. The method of claim 9 wherein Z is spaced from the carbon bonded to X by ═CR⁶—, wherein R⁶ is H or linear, branched or cyclic alkyl (1-6C).

11. The method of claim 9 wherein K is a substituted or unsubstituted carbocyclic aromatic system.

12. The method of claim 11 wherein K is p-fluorophenyl.

13. The method of claim 4 wherein Y is of formula (8).

14. The method of claim 13 wherein K is substituted pyrrole.

15. The method of claim 14 wherein said substitutions comprise substituted or unsubstituted aromatic systems.

16. The method of claim 15 wherein the substitutions comprise alkyl (1-6C) and alkoxy (1-6C).

17. The method of claim 1 wherein said statin-like compound is atorvastatin, cerivastatin, lovastatin, mevastatin, simvastatin, fluvastatin, pravastatin, rosuvastatin or NK-104 in hydrolyzed or unhydrolyzed form.

18. The method of claim 1 wherein the statin-like compound is apamine or zaragozic acid.

19. The method of claim 1 wherein said nitric oxide generating system comprises an organic NO donor.

20. The method of claim 19 wherein said organic NO donor is glycerol trinitrate, isosorbide mononitrate, isosorbide dinitrate, erythrityl tetranitrate, pentaerythritol tetranitrate, or L-arginine.

21. The method of claim 20 wherein said organic NO donor is glycerol trinitrate or L-arginine.

22. The method of claim 1 wherein the nitric oxide-generating system comprises an NO synthesizing enzyme.

23. The method of claim 22 wherein the NO synthesizing enzyme is one or more isoforms of NO synthase and/or mitochondrial aldehyde dehydrogenase.

24. The method of claim 23 wherein said enzyme is provided as its encoding DNA.

25. The method of claim 24 wherein said encoding DNA is contained in a viral vector or in cells obtained from the subject or is naked DNA.

26. The method of claim 1 wherein said nitric oxide generating system comprises an agent that activates an NO-synthesizing enzyme or enhances the production thereof.

27. The method of claim 26 wherein said agent is cyclosporin A, FK506, felodipine, nicorandil, nifedipine, diltiazem, resveritrol, sapogrelate or quinapril.

28. The method of claim 1 wherein the PDE inhibitor is a nonspecific PDE inhibitor.

29. The method of claim 28 wherein said inhibitor is caffeine, theophylline, pentoxifylline, or 3-isobutyl-1-methylxanthine.

30. The method of claim 1 wherein the PDE inhibitor is specific for one or two phosphodiesterase families.

31. The method of claim 30 wherein said PDE inhibitor is dipyridamole, MY-5445, sildenafil, Zaprinast™, or rolipram.

32. The method of claim 1 wherein said two components comprise at least one nitric oxide generating system and at least one statin-like compound.

33. The method of claim 1 wherein the two components comprise at least one statin-like compound and at least one phosphodiesterase inhibitor.

34. The method of claim 1 wherein the two components comprise at least one nitric oxide generating system and at least one phosphodiesterase inhibitor.

35. The method of claim 1 wherein said two components are co-administered.

36. The method of claim 35 wherein said two components are co-administered in a single composition.

37. The method of claim 1 wherein said two components are administered sequentially.

38. The method of claim 1 wherein said subject is characterized by a condition selected from the group consisting of osteoporosis, bone fracture or deficiency, primary or secondary hyperparathyroidism, periodontal disease or defect, metastatic bone disease, osteolytic bone disease, post-plastic surgery, post-prosthetic joint surgery, and post-dental implantation.

39. The method of claim 1 which further comprises administering to said subject one or more additional agents that promote bone growth or that inhibit bone resorption.

40. A pharmaceutical composition in unit dosage form to enhance bone formation in a vertebrate animal which composition comprises a pharmaceutically acceptable excipient and an amount, effective to promote bone formation, of at least two of the following components
a) at least one nitric oxide generating system;
b) at least one statin-like compound; and
c) at least one phosphodiesterase inhibitor.

41. The composition of claim 40 wherein the statin-like compound is atorvastatin, cerivastatin, lovastatin, mevastatin, simvastatin, fluvastatin, pravastatin, rosuvastatin or NK-104 in hydrolyzed or unhydrolyzed form.

42. The composition of claim 40 wherein said nitric oxide generating system comprises an NO donor.

43. The composition of claim 42 wherein said NO donor is glycerol trinitrate or L-arginine.

44. The composition of claim 41 wherein the phosphodiesterase inhibitor is caffeine, pentoxifylline, theophylline, or 3-isobutyl-1-methylxanthine.

45. The composition of claim 40 which comprises at least one nitric oxide generating system and at least one statin-like compound.

46. The composition of claim 40 which comprises at least one nitric oxide generating system and at least one phosphodiesterase inhibitor.

47. The composition of claim 40 which comprises at least one statin-like compound and at least one phosphodiesterase inhibitor.
